(19) United States
(12) Patent Application Publication
(10) Pub. No.: US 2008/0125797 A1
(43) Pub. Date: May 29, 2008

(54) Methods and Devices for Organ Partitioning

(76) Inventor: Brian Kelleher, Del Mar, CA (US)

Correspondence Address: SCOTT EVANS, 1252 COUNTRY HILLS DR., SANTA ANA, CA, 92705, US

(21) Appl. No.: 11/944,767

(22) Filed: Nov. 26, 2007

Related U.S. Application Data
(60) Provisional application No. 60/867,332, filed on Nov. 27, 2006.

(51) Int. Cl. A61B 17/11; A61B 17/34

(52) U.S. Cl. 606/153; 606/185

(57) ABSTRACT

Methods and devices for partitioning hollow organs or cavities of the body are described. A tissue securement device having securement arms is positioned through portals formed in opposing tissue walls of an organ. The securement arms may be articulated to facilitate placement. Once positioned the securement arms may be activated to secure the opposing walls of the organ together. The securement arms may secure the walls together through the use of staples, t-tags, sutures, clamps, helical anchors, braided anchors or fastening devices or systems.

### CLAIM OF PRIORITY

This application claims priority under 35 U.S.C. § 119(e) to U.S. Provisional Application No. 60/867,332, filed Nov. 27, 2006, the entire contents of which are hereby expressly incorporated by reference.

### BACKGROUND OF THE INVENTION

1. Field of the Invention

The present invention relates to devices and methods for partitioning a hollow organ of the body. In particular positioning a device through a natural orifice, forming openings in the walls of the organ, positioning a device through these openings and securing the walls of the organ together.

2. Description of the Related Art

Hollow organs or cavities in the body are sometimes secured together in order to create compartments within the hollow organ or to reduce the volume of the organ. These devices are typically delivered to the hollow organ using open surgical or laparoscopic means. For example, the stomach may be partitioned as a treatment for GERD (gastro esophageal reflux disease) or obesity. In these types of operations, the partitioning may create a smaller gastric volume so that the patient will eat less before reaching satiety. Often the walls of the stomach are stapled together during gastric reduction surgery along certain meridians to compartmentalize the stomach.

Preferably a device used to compartmentalize an organ such as the stomach is configured for delivery through a natural orifice of the body using flexible endoscopic means. Examples of these orifices are the esophagus, anus, vagina or bladder. It is generally believed that interventions to a patient's body through one of these orifices may be less traumatic for the patient, decrease surgical time, decrease post-operative care and potentially reduce costs as compared to open or laparoscopic surgery which require the creation of “unnatural” cuts or orifices in the body.

Despite the benefits to the patient, endoscopic partitioning of organs through natural orifices is difficult. This is primarily because the natural orifices present small volumes where working space is limited; therefore, the placement of staples, t-tags, sutures or other fastening systems is difficult from inside the organ or cavity. Particularly in the case of gastric stapling, placing a stapler inside the stomach and then securing two opposing walls of the stomach together is difficult when working from only inside the stomach without having access to the inside and the outside walls of the stomach. It is possible to pull in linear tucks of stomach wall tissue from two opposing walls and to then staple or otherwise affix such segments together, but it has been shown that such couplings are less apt to heal together and therefore hold up over time than couplings that involve stapling the full thickness of the two opposing walls from outside the stomach. The present invention may overcome the limitations of typical flexible endoscopic stapling or suturing systems by positioning a portion of the securement mechanism outside the organ wall so that the full thickness of both walls may be joined together. By joining the full thickness of both walls, and by joining the walls from outside the organ, the present invention may provide a more reliable and robust partitioning solution.

Devices that can be introduced into a hollow body organ such as the stomach through natural orifices and secure the walls of the stomach together easily and securely are needed. In particular a device that can secure the walls of the stomach together using a portion of the outside stomach wall in order to at least partially secure opposing walls of the stomach together to partition the stomach into compartments would be beneficial.

### BRIEF SUMMARY OF THE INVENTION

The preferred methods and devices described herein provide for improved methods and devices for tissue securement, and, in particular, for securing the walls of a hollow organ together. Such securement may be useful for creating partitions within the hollow organ such as for the treatment of obesity involving the stomach. The device may be positioned in the body with the aid of an endoscope and may be used from within the hollow organ to bring the organ walls together easily so that the walls may be secured.

A first aspect of the invention is a method for creating a partition within a hollow organ of a patient. A device may be advanced from outside the patient through a natural orifice of the patient and into the hollow organ where at least one opening may be formed in each wall of the organ. A tissue securement element that has securement arms attached may be at least partially advanced through these openings to position the opposing walls of the organ between the securement arms. The securement arms may be activated to secure the opposing walls together. The securement arms may secure the walls together through the use of staples, t-tags, sutures, clamps, helical anchors, braided anchors or fastening devices or systems.

A further aspect of the invention is a method of creating the openings in the tissue by penetrating the organ wall with a puncturing element. The puncturing element may be a combination of a needle, knife, radio frequency electrode or dilatation balloon. This aspect may further include suctioning a portion of the wall into a cavity in the first device prior to the creation of the opening.

In another embodiment of the invention the securement of tissue may be aided by modifying the tissue properties of at least one of the organ walls. These modifications may include cutting, piercing, thermal injury, chemical injury, excision or crushing.

In another embodiment of the invention the method may include repositioning the securement arms through the openings in the organ wall to secure the organ walls together at one or more additional locations in the organ.

Another aspect of the invention is an apparatus for creating a partition within a hollow organ through a natural orifice. The apparatus may include a distal portion having a puncturing element and a tissue securement element with the securement element designed to secure two walls of the organ together. The apparatus may include an elongate portion that extends from the distal portion to a proximal portion and the elongate portion may be sized to extend from the hollow organ, through the natural orifice, to outside the patient. The proximal portion may include a controller to activate the puncturing element or the securement element. The securement element may utilize securement arms that are linked to each other at a pivot point. These arms may use an actuating member that causes the securement arms to pivot and move closer together or farther from apart from each other.

In another aspect of the invention the puncturing element mat be retractably positioned at the end of the securement arm such that the puncturing element can be extended to puncture tissue and retracted to prevent additional tissue puncture. In this aspect the puncturing element may be integral to the securement arm and may utilize an actuator to extend or retract the puncturing element.

In another aspect of the invention the securement element may have a coupling element nested in one of the securement arms. The coupling element may be used to pierce the tissue and couple to a mating latching element disposed in the other securement arm. The coupling element may be pulled using an actuating wire which may cause the securement arms to forcibly press against one another to modify the characteristics of the tissue pinched in between.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a schematic view of a stomach illustrating tissue securement positions of the prior art.

FIG. 1B is a schematic view of the stomach illustrating a preferred line of securement of the stomach walls.

FIG. 2A is a schematic view of an endoscope with an electrocautery needle positioned to make a puncture the stomach wall.

FIG. 2B is a section view of an alternate tissue puncturing embodiment.

FIG. 3A is a schematic view of an esophageal tract and stomach showing a method of positioning the securement device using guidewires.

FIG. 3B is a perspective view of the securement device positioned over guidewires.

FIG. 3C is a side view of the device shown in FIG. 3B.

FIG. 3D is a perspective view showing the device of FIG. 3B positioned across the stomach walls (shown in section).

FIG. 3E is a securement device with an integrated puncturing element.

FIG. 4A is a schematic view of the range of preferred securement for a device placed through a portal.

FIG. 4B is a schematic view of an embodiment of a pivotable securement device.

FIG. 4C is a schematic view of the range of use for the pivotable securement device of FIG. 4B.

FIG. 4D is a schematic view of a preferred use of the pivotable securement device of FIG. 4B.

FIG. 5A is perspective view of a securement device capable of pinching tissue.

FIG. 5B is a view of the device of FIG. 5A with a spreading force created by a pusher element.

FIG. 5C is a view of the device of FIG. 5A with a spreading force created by a pneumatic/hydraulic mechanism.

FIG. 6 is a schematic view of the device of FIG. 5A showing a coupling element.

FIG. 7A is a schematic view of a tapered sliding hinge joint.

FIG. 7B is a schematic view of a tapered sliding hinge joint when the jaws first clamp around tissue.

FIG. 7C is a schematic view of a tapered sliding hinge joint which has been modified to increase the clamping force of the proximal portion of the securement arm.

### DETAILED DESCRIPTION OF THE INVENTION

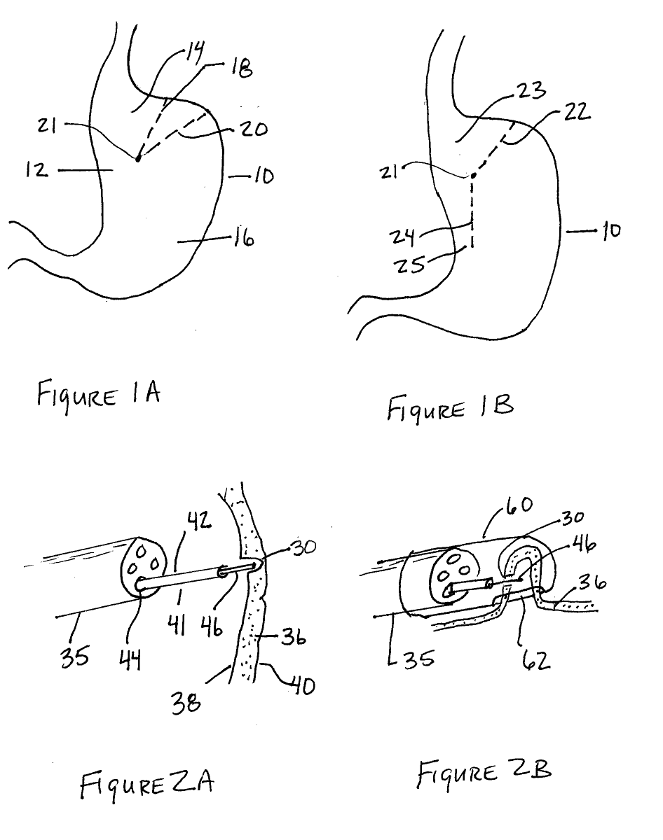

FIGS. 1-7 depict various embodiments of an organ partitioning device that may be used to secure walls of a hollow organ together in order to create compartments within the hollow organ or to reduce the volume of the organ. The described device may be delivered to the hollow organ with a number of methods including percutaneous, surgical and endoscopic means. Preferably the device is configured for delivery through a natural orifice of the body using flexible endoscopic means.

The device and method of the present invention may be applicable to many body organs, cavities, lumens or vessels and may be used to treat a wide variety of indications where a secure wall to wall requirement is present. The coupling of one wall of a body lumen to another may be useful in any number of disease states, treatment modalities and body sites. Although this invention and method may be illustrated in this description using the stomach, this is not meant to be limiting in any way. It is anticipated that the invention so described may be used in many areas of the body such as vessels, ducts, cavities, spaces to join tissue together from and for many reasons. Likewise, although the delivery of the device through a patient's mouth is illustrated, it is anticipated that the device could be delivered through any body orifice or even percutaneously to a targeted interventional site.

In FIG. 1A a stomach 10 is shown having a lesser curvature 12, cardia 14 and an inner cavity 16. The cardia 14 and the lesser curvature 12 are landmarks that the surgeon may use to insert a line of staples to create a reduction in the stomach volume. A line of staples is often placed along line 18 or along line 20 from a point 21 to form compartments in the stomach of various sizes. The length and the direction of these securement lines may vary patient to patient. As shown in FIG. 1B, lines of staples may be extended or separate securement lines may be combined to extend the length of the compartment. As shown, line 22 may be created to form a small digestive pouch 23 that acts as a small stomach and line 24 may be created to form a narrow exit passageway 25 from the compartment 23. The combination of these compartments may be useful in the treatment of obesity by reducing the size of the digestive space and to slow the evacuation of the contents of this smaller pouch through the restricted passageway 25.

In one embodiment of the invention broadly described is a method and device to form unions of one wall of the stomach to an opposing wall thus creating wall-to-wall securements that may be positioned along the lines described. The method teaches the creation of portals in the wall of the stomach. Referring to FIG. 2A-B, these portals 30 are formed in the wall 36 of the stomach 10 and extend from an inner wall 38, through the wall 36 and exit through the outer wall 40. Theses portals are created so that a portion of the securement device may be inserted into these portals and through the wall of the stomach 36 to access the outside surfaces of the stomach. The formation of these portals 30 may be accomplished using a variety of methods known in the art for forming a hole in tissue. Preferably the portal is large enough to accommodate the device placed through it. However the portal 30 size should not be so large that it is difficult to close the portal 30 once the device is removed or permits the leakage of stomach contents around the securement device during the procedure.

As shown in FIG. 2A, a puncturing element 41 for portal formation may be delivered to the target site using the working lumen 44 of an endoscope 35. The puncturing element may be retracted inside the working lumen 44 of the endoscope 35 until the target site is near. Then under endoscopic guidance, the puncturing element 41 may be deployed from the working lumen to create the portal 30. The puncturing element in FIG. 2A is an electrocautery device 41 that has an outer sheath 42 surrounding a monopolar or bipolar electrocautery needle 46. The needle cuts through tissue by using heat to cauterize and destroy tissue near the tip of the needle 46. The needle 46 forms a portal 30 as the tissue is eliminated and the needle 46 is advanced. Continued deployment causes the needle cautery device to completely pierce the wall 36. The wall of the stomach may close back down if the puncturing element 41 is removed without protection so a guidewire may be inserted through the tip of the needle 46 or alongside the needle 46 to mark the location of the hole. The needle should not be deployed much beyond the outer wall 40 so that surrounding tissue or organs are not affected. In an alternate embodiment of the method, the portal may be secondarily enlarged with the use of an obturator, dilatation balloon, expansion mesh or basket or other device used to enlarge tissue openings. In a preferred embodiment of the device at least two portals should be created so the puncturing element 41 may be relocated to a site preferably opposite to the first portal and a second portal formed in the opposing wall.

An alternate method of a creating a stomach wall opening is shown in FIG. 2B. An end cap 60 is positioned about the end of an endoscope 35. The end cap 60 has a closed distal end and the proximal end is tightly fitted to the outside diameter of the endoscope 35. An opening 62 is located on one side of the end cap 60. The endoscope is introduced into the stomach and suction is applied through one of the working lumens. As the opening is positioned against the stomach inner wall 38, the suction draws the stomach wall 36 into the opening 62. Once the stomach wall is inside the end cap 60, a puncturing element 41 can be advanced to form a portal through the tissue wall 36. In an alternate embodiment of the method (not illustrated), portals may be formed simultaneously through both walls (anterior and posterior) by having two opposing openings 62 on end cap 60, positioned so as to draw the opposing walls into end cap 60 and then to puncture each wall with two separate puncturing elements 41. This technique would potentially save time by eliminating the need to reposition a puncturing device to form both portals.

The port locations are preferably directly opposed from each other with one portal on the anterior stomach wall and one portal on the posterior stomach wall. However various locations and configurations of portals may be used and the use of such is anticipated.

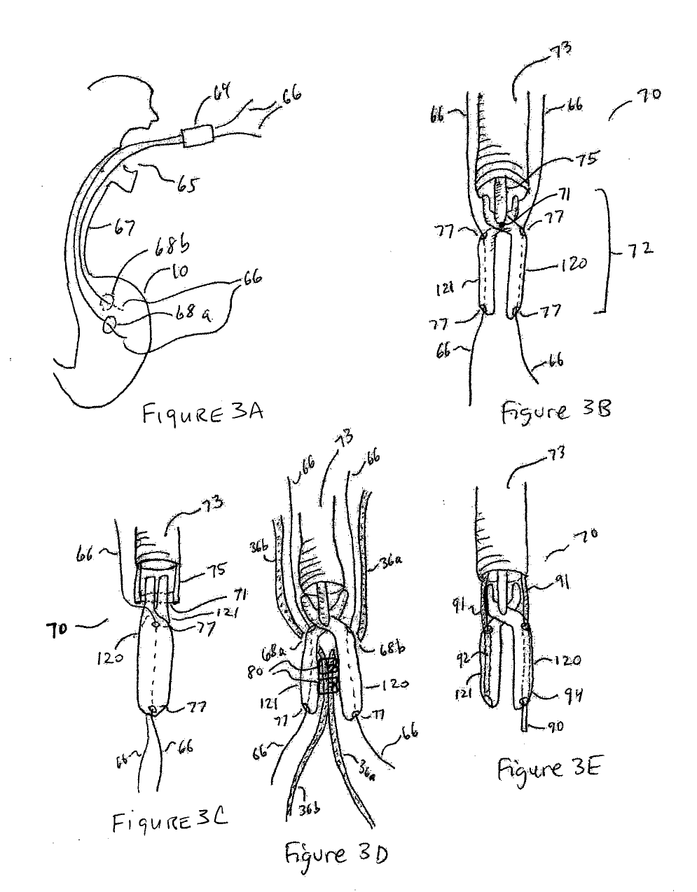

Following the portal formation, a guidewire may be placed through the portal to again preserve the opening and to guide additional devices through the portal as shown in FIG. 3A. A partition forming device 64 is shown prepared for introduction through a patient's mouth 65 along guidewires 66. The guidewires traverse the patients esophagus 67 and into the stomach 10. The guidewires 66 traverse the posterior portal 68a and the anterior portal 68b and extend outside the stomach wall. As can be understood, as device 64 is moved along the guidewires 66, at least a portion of device 64 may traverse the stomach wall and be positioned outside the stomach wall.

One embodiment of the invention is described in FIG. 3B. A partition forming device 70 has a distal portion 72 and an elongate portion 73. The elongate portion 73 extends from the distal portion 72 of the device 70 to a proximal portion of the device 70 (not shown) which is outside the patient's body. The distal portion 72 has two securement arms or jaws 120 and 121 that are used to secure the tissue as will be described later. The arms are connected at pivot 71 so that the arms may move about the pivot 71 to increase or decrease the distance between the arms. Pivot 71 is supported by pivot support 75, which is linked to elongate portion 73. Elongate portion 73 may consist of an outer sheath made up of helically-coiled wire or may be an extruded tubular body, as is known to those skilled in the art of endoscopic device design. Guidewires 66 are shown extended through guidewire channel apertures 77, which provide a guide channel for device 70 to ride along guidewires 66.

FIG. 3C is a side view of device 70 showing more detail of the embodiment. In particular, a possible arrangement of arms 120 and 121 around pivot 71 is shown, along with a possible configuration of pivot support 75.

FIG. 3D depicts the embodiment of FIGS. 3B and 3C with arms 120 and 121 shown traversing portals 68a and 68b. By way of example, portal 68a may be formed in the anterior stomach wall and portal 68b may be formed in the posterior wall of the stomach. Guidewires 66 extending through guide channel apertures 77 provide the pathway for advancing device 70 such that arms 120 and 121 extend through portals 68b and 68a, respectively.

Once the arms 120 and 121 are positioned through the portals 68a-b and are aligned along the meridian of choice as shown in FIGS. 1A-B, the arms may be brought closer together so that a securement element positioned on the arms may be activated. The securement element may be any one of a number of securement devices known to those skilled in the art. For example, the securement element may be a stapler with a driver positioned on one arm and with an anvil positioned on the other arm. Alternately, the securement element may be a driver for t-tags, a suturing device, a clamp, a helical or braided anchor, a heat source or tissue welder.

The securement element is configured to secure one tissue wall 36a to an opposing tissue wall 36b. As shown the securement element in FIG. 3D is a stapler and staples 80 are shown traversing from arm 121, across two layers of tissue 36a-b and back to arm 120. In this way the two layers of tissue can be secured together using a device that can be deployed through a natural body orifice without requiring open surgery. The activation of the securement element may be directed by an operator from outside the patient's body using activation mechanisms such as pull/push wires, spring loaded delivery or other commonly used techniques and devices.

In another embodiment of the invention, the puncturing element may be integrated into the arms of the partition forming device 70. As illustrated in FIG. 3E, partition forming device 70 is shown with arms 120 and 121. In the distal end portion 94 of arm 120 a puncturing element 90 is housed and is shown in an extended position. The puncturing element 90 may be comprised of an electrocautery tip, a knife, a needle, radio frequency electrode, dilatation balloon or any other suitable tissue portal forming technology. The puncturing element 90 may be housed inside a lumen formed inside the arm 120 and may be extended and activated and later retracted using an activator operated from outside the body. Puncturing element elongate body 91 is shown extending from puncturing element 90 through a lumen in arm 120 and then extending within elongate portion 73 of device 70. A puncturing element 92 is shown retracted inside arm 121. The incorporation of portal cutting into a partition forming device may have several advantages. First because the two components are housed in a single device, the portal forming and partition formation can occur quickly. The number of device exchanges is reduced potentially reducing patient discomfort and procedure length. The need for guidewire insertion to maintain the patency of the portal is also eliminated because the portal formation and arm insertion occur at the same time.

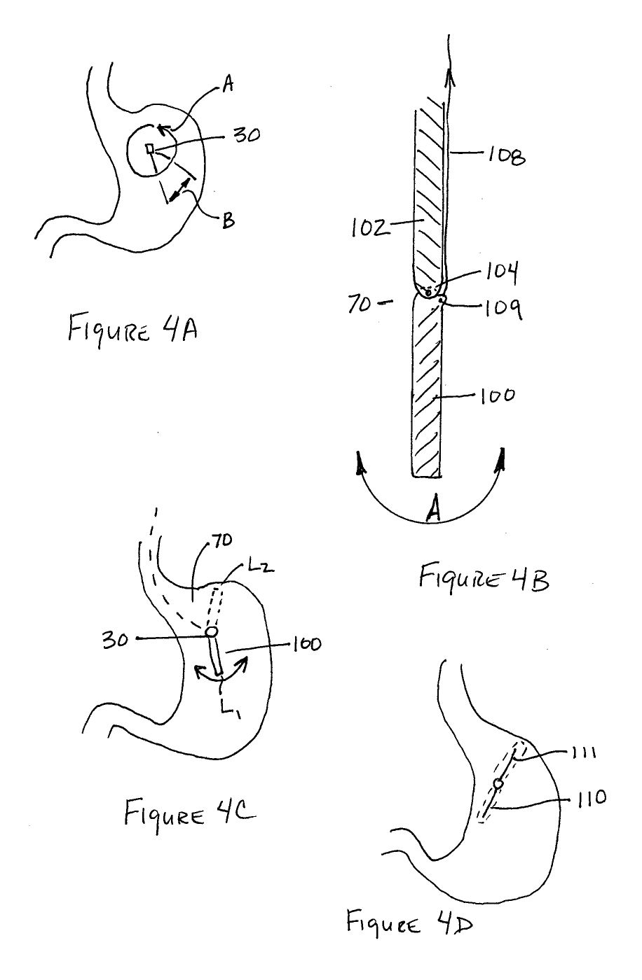

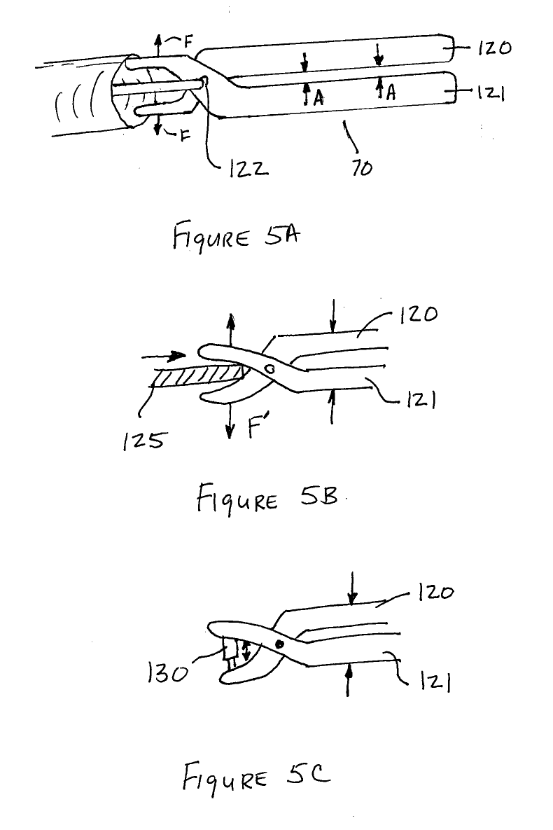

The methods for forming a line of wall-to-wall tissue securement according to the present invention are illustrated in FIGS. 4A-C. As shown in FIG. 4A, a portal 30 has been formed in a wall of a stomach, and the possible locations of tissue securement created by a partition forming device are as shown by the 360° arc labeled A. However if the body of the partition forming device is not articulated, then the securement possibilities are limited. As a rigid device is advanced through the portal 30, only a limited range of locations shown by the arc labeled B would be feasible, and this may not be provide for an optimal location of a securement line. FIG. 4B illustrates a partition forming device 70 having an articulated body where the distal portion 100 of device 70 is joined to the main body 102 with a hinged pivot 104. The hinged pivot 104 permits the securement element to be introduced into the patient in a straightened configuration and then the distal portion 100 may be rotated about the pivot 104 to a different position relative to the main body. The rotation of the distal portion 100 about the pivot 104 is shown as arrow A. The articulation of the distal portion 100 may be directed by the activation of an activation wire 108 attached to an offset anchor point 109 at its distal end and extending outside the patient's body for activation by an operator. The hinged pivot may be spring loaded so that as the distal portion 100 is rotated by pulling the activation wire 108 the spring is loaded so that a corresponding relaxation of the wire 108 causes the spring to return the distal portion 100 to its original position. In another embodiment, a secondary offset anchor point may be used with another activation wire so that the distal portion 100 may be articulated in either direction of arrow A by a combination of pulling or relaxing the activation wires. In a further embodiment, activation wire 108 may extend through a tubular casing affixed to the distal end of body 102, such that pulling on wire 108 causes distal portion 100 to pivot in one direction, and pushing on wire 108 causes distal portion to pivot in the opposite direction. It will be understood that such tubular casing may be a separate element or it may be a feature of body 102.

Referring to FIG. 4C, a partition forming device 70 may be introduced into the patient in a straightened configuration to facilitate insertion through the esophagus, into the stomach and through the portal 30 formed in the stomach wall to a first location L1. The straightened configuration may help positioning the partition forming device 70 across the portal 30. Once through the portal 30, however, the securement element 100 may be rotated around the pivot 104 (see FIG. 4B) as shown by the arrows in FIG. 4C to a different location such as the one indicated by L2. Alternately, the partition forming device 70 may be introduced into the esophagus in a straightened configuration and then rotated to the desired angle in the stomach and then subsequently inserted through the stomach wall in a rotated position. As can be appreciated, the addition of an articulated securement element greatly increases the potential number of intervention angles and associated treatment sites possible.

As shown in FIG. 4D, the articulated securement element also facilitates linking multiple securement lines together to form a longer securement line then would be possible without articulation. As shown, a first securement line 110 may be formed in one direction and then a second securement line 111 may be formed at a different angle or direction than line 110. The combination of these securement lines may not only create a longer partition, but may also allow the creation of multiple compartments, such as the smaller gastric pouch and restrictive passageway described in FIG. 1B.

In addition to a primary securement of the walls of a hollow organ, it may be beneficial to utilize a secondary method to enhance or reinforce the primary securement. Modifying the securement site may increase the strength of the securement and prevent premature failure of a securement. The site may be modified by introducing scarring or tissue irritation to the securement site. This scarring may modify the mechanical properties of the securement site so that the chance of anchor pull-out is reduced. Additionally, the injury may accelerate a healing response that may improve wall-to-wall healing and subsequent strength.

One aspect of the invention is to modify the securement site by crushing the tissue at the site. This may be accomplished by applying a crushing force to the tissue directly under the securement arms. This force may be applied by utilizing the pivot/arm design described previously. Referring to FIG. 5A, a partition forming device 70 is shown with securement arms or jaws 120 and 121 joined at pivot 122. In order to apply a greater clamping force to tissue positioned between the arms or jaws 120 and 121, a greater actuation force, as shown by arrows F needs to be applied. The reactionary force along the tissue interface surfaces of jaws 120 and 121 is shown by arrows labeled A. One embodiment of the invention is illustrated in FIG. 5B where the application of a pusher element 125 near the pivot 122 can increase the clamping force. As the pusher element 125 is moved distally along the longitudinal axis of the device 70, the distal portion of the pusher element contacts a portion of the jaws that is proximal of the pivot. In response to this application of force, the proximal portion of the jaws are spread apart in the direction F′. As the proximal portion of the jaws move in direction F′, the distal portion of the arms are forced together with increased clamping force. If the force is high enough, tissue will be crushed by the distal portion of the securement arms or jaws 120 and 121.

In another embodiment of the invention, a hydraulic or pneumatic element 130 is positioned in between the proximal end portions of the securement arms. This element is positioned so that when the hydraulic/pneumatic element 130 is activated, a force is directly applied to the proximal end portions of the securement arms which in turn drives the distal portions of the securement arms together. As these arms are forced together with increased clamping force, tissue that is situated in between the arms will be crushed. This tissue crushing may increase the likelihood of irritating the tissue and triggering a healing response that will increase the probability that the securement will not pull out of the stomach wall and that the partitioning will be secure.

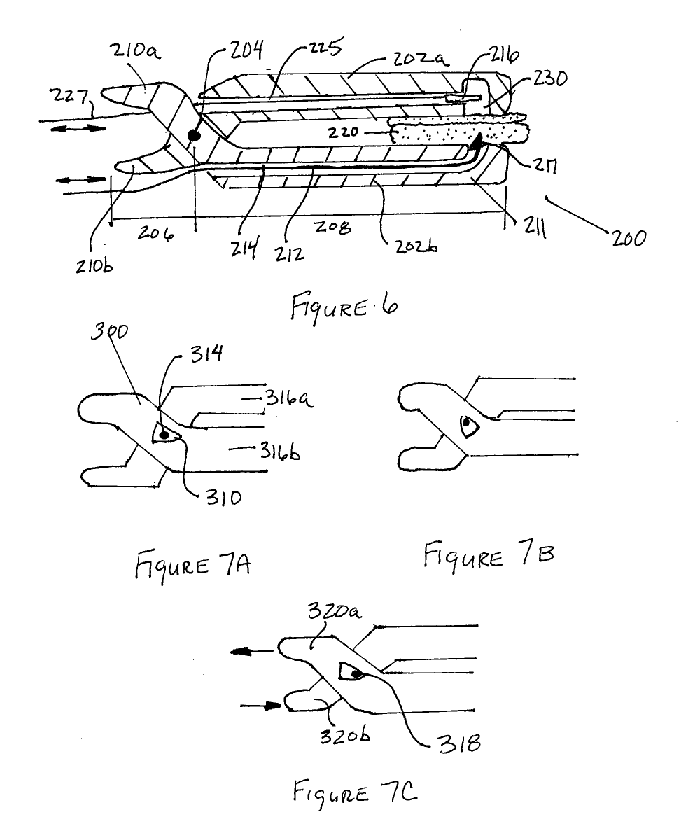

Another embodiment of the device that may be used to sustain increased crushing loads on affected tissue is shown in FIG. 6. A securement device 200 is shown having two securement arms 202a and 202b. These arms 202a-b may be used to crush and secure two walls of a hollow organ or cavity similarly to securement device 70 as previously described. The device 200 has a proximal portion 206 and a distal portion 208 with portion 208 having the securement arms 202a-b and the proximal portion 206 having the actuating arms 210a and 210b. The arms 202a-b are coupled together at pivot 204 and can be opened and closed by manipulating the actuating arms 210a-b about the pivot 204. Securement arm 202b has at least one coupling element 211 which is designed to couple the two securement arms 202a-b together so as to exert a tissue crushing force on tissue positioned in between the two securement arms 202a-b. The coupling element comprises an actuating wire 212 that extends from the end of arm 202b along an elongate portion (not shown) of the device 200 and outside the patient's body. The wire 212 has a barb 217 that is designed to couple with a retractable tang 216 located on the opposing securement arm 202a. Securement arm 202b has a channel 214 that extends along the length of the securement arm 202b into which actuating wire 212 is positioned. The actuating wire 212 can be moved inside this channel 214 by an operator. When the wire 212 is extended, it is directed through at least two layers of tissue 220 and into an opposing cavity 230 of arm 202a. A channel 225 houses the retractable tang 216 and extends along the length of the securement arm 202a. Retractable tang 216 is attached to a retracting wire 227 that extends from the end of arm 202a along an elongate portion (not shown) of the device 200 and outside the patient's body. The wire 227 is designed to retract the tang 216 after coupling with the actuating wire 212 is complete and wire 212 has been pulled in order to crush tissue layers 220. In one embodiment the tang 216 is spring loaded so that it extends into cavity 230 in a resting condition and then can be retracted using retracting wire 227 for de-coupling the actuating wire 212. Even though only one coupling element 211 is shown, it is anticipated that more than one coupling element 211 could be integrated into device 200 so that the crushing force exerted upon the tissue layers 220 could be uniform along the securement arms 202a-b.

When the securement device 200 is deployed about two layers of tissue 220, the securement arms 202a-b may be closed about the tissue 220 by manipulating the actuating arms 210a-b located in the proximal portion 206. The coupling element 211 may then be actuated by extending the actuating wire 212 which forces barb 217 at the end of the actuating wire 212 through tissue 220 and into the cavity 230. The barb 217 couples to the extended tang 216 by latching the barb edge over the tang. The two securement arms 202a and 202b are now coupled together. As can be seen, as the actuating wire is pulled in a proximal direction, the barb, which is coupled to the retractable tang 216 exerts a force on the tissue 220 disposed between the securement arms. This force is intended to be sufficient to crush the tissue and initiate an inflammatory response as previously described. The securement arms 202a-b may have additional securement elements such as a stapler that can also be used to secure the tissue layers 200 together. These securement elements may be deployed either before or after the tissue crushing procedure described. The retractable tang 216 may be retracted away from the cavity 230 using the retracting wire 227 which de-couples the tang 216 and the barb 217 and securement arm 202a from securement arm 202b. The actuating wire 212 may be similarly withdrawn so that the barb is withdrawn from the tissue 220 and back into the channel 214.

With a tissue crushing design as shown in securement device 200, it is possible that much of the crushing force is exerted at the distal end of the securement arms and less at the region close to the pivot. An adjustable pivot 300 is shown in FIGS. 7A-C that may distribute the crushing force more evenly along the entire length of the securement arms. The adjustable pivot 300 has a sliding hinge joint comprised of a hinge hole 310 and a hinge pin 314 as shown generally in FIG. 7A. The adjustable pivot 300 is designed to loosely couple the securement arms 316a and 316b in a first condition and then adjust to a second coupling position as a tissue crushing force is applied to the tissue using the securement arms 316a-b. When the securement arms 316a-b first clamp around the tissue, the adjustable pivot is loose as shown in FIG. 7B. As shown in FIG. 7C, as the securement arms 316a-b are manipulated by their actuating arms 320a-b to force the securement arms together, one securement arm is also pulled more proximally relative to the other. This action forces the hinge pin 314 into a narrow portion 318 of the hinge hole 310. This adjustment of the pivot point increases the crushing force of the two securement arms close to the pivot point so that the crushing force may be more evenly distributed along the length of the securement arms 316a-b. This may allow a more even crush of the tissue in between the securement arms to provide a uniform modification of the tissue properties along the securement line.

Once the securement devices described have modified the tissue by crushing the tissue and/or secured the tissue layers with one or more fastening devices or methods, the securement device may be removed from the tissue portal and withdrawn into the stomach and removed from the patient. The remaining portal may self close on its own or the portal may be closed by using a stitching device such as the EndoCinch® by Bard or by using a clipping device or other means to close the portals in the tissue.

The securement arms may also incorporate a cutting element in addition to a securement element that can be used to cut the tissue after the fastening elements such as staples are applied. Such cutting action, which may be partial or full-thickness, has been known to further enhance the desired healing response.

Although this invention has been disclosed in the context of certain preferred embodiments and examples, it will be understood by those skilled in the art that the present invention extends beyond the specifically disclosed embodiments and/or uses of the invention and obvious modifications and equivalents thereof. Thus it is intended that the scope of the present invention herein should not be limited by the particular disclosed embodiments described above, but should be determined only by a fair reading of the claims that follow.

What is claimed is:

1. A method for creating a partition within a hollow organ of a patient, said hollow organ having two opposing walls, comprising the steps of:
a. passing a device from outside the patient through a natural orifice of the patient into the hollow organ,
b. creating at least one opening in each wall of the hollow organ,
c. passing a tissue securement element having first and second securement arms at least partially through said openings so as to position the opposing walls between the first and second securement arms at a first location,
d. activating said first and second securement arms to secure the opposing walls together.

2. The method of claim 1 wherein said hollow organ is the stomach.

3. The method of claim 2 wherein said natural orifice is the mouth and esophagus.

4. The method of claim 2 wherein said natural orifice is the vagina or anus.

5. The method of claim 2 wherein the secured opposing walls form a first partition in the stomach.

6. The method of claim 5 wherein at least a portion of the first partition is positioned near the cardia region of the stomach.

7. The method of claim 5 wherein at least a portion of the first partition is positioned near the lesser curvature region of the stomach.

8. The method of creating at least one opening as in claim 2 further comprising penetrating the organ wall with a puncturing element, wherein said element comprises one or a combination of a needle, knife, radio frequency electrode or dilatation balloon.

9. The method of claim 8 wherein a portion of the wall is suctioned into a cavity in the first device prior to the creation of the opening.

10. The method of claim 1 wherein the first and second securement arms secure the walls together through the use of one or a combination of staples, t-tags, sutures, clamps, helical anchors or braided anchors.

11. The method of claim 1 including modifying the tissue properties of at least one of the opposing walls.

12. The method of claim 11 wherein the modification includes cutting, piercing, thermal injury, chemical injury, excision or crushing.

13. The method of claim 1 further comprising repositioning the first and second securement arms through the at least two openings so as to position the opposing walls between the first and second securement arms and activating said first and second securement arms to secure the opposing walls together at a second location.

14. The method of claim 13 wherein the secured opposing walls form a second partition in the stomach.

15. The method of claim 14 wherein said first partition is created near the cardia, thereby forming a pouch, and wherein said second partition is created near the lesser curvature, thereby forming a tubular outlet to the pouch.

16. The method of claim 15 further comprising removing the securement arms from the openings and closing the openings.

17. An apparatus for creating a partition within a hollow organ having two opposing walls comprising:
a distal portion having at least one puncturing element and at least one tissue securement element, said securement element configured to secure two walls of the organ together, an elongate portion extending from the distal portion to the proximal portion and sized to reach from the hollow organ, through the natural orifice, to outside the patient, the proximal portion including at least one controller to activate the at least one puncturing element or at least one securement element.

18. The apparatus of claim 17 wherein said puncturing element is a needle, knife or an electrode capable of delivering radio frequency energy.

19. The apparatus of claim 17 wherein said securement element comprises a first and a second securement arm linked to each other at a pivot point and having an actuating member extending from the distal portion to a controller located in the proximal portion of the device, such that actuation of the actuating member causes the first and second securement arms to pivot and thereby move closer or farther from each other.

20. The first and second securement arms of claim 19 further comprising a stapler, t-tag applier, clip applier, clamp applier, suturing device, helical anchor applier or tissue welder.

21. The apparatus of claim 17 wherein said puncturing element comprises an electrode which is energized with radio frequency energy and said tissue securement element comprises a stapling device.

22. The apparatus of claim 17 wherein the puncturing element is retractably positioned at the end portion of the securement arm such that the puncturing element can be extended to puncture tissue and retracted to prevent additional tissue puncture.

23. The puncturing element of claim 22 further comprising an actuator extending from the distal portion to a controller located in the proximal portion of the device, said actuator enabled to extend or retract said puncturing element.

24. The securement element of claim 20 further comprising at least one coupling element nested in one of the two securement arms and having an actuating wire extending to the proximal portion of the device, such that when the securement arms pivot and the actuating wire is advanced, the coupling element pierces the tissue and couples to a latching element in the other securement arm such that pulling on the actuating wire causes the securement arms to forcible press against one another thereby crushing any tissue positioned between the securement arms.

25. The apparatus of claim 24 wherein said securement arms contain staple and anvil components to deploy and shape staples through the opposing walls of the hollow organ.

26. The apparatus of claim 25 wherein said securement arms apply a crushing force across the portion of the two opposing walls into which the staples are deployed.

27. The apparatus of claim 21 wherein said stapling device deploys multiple rows of staples and includes a cutting element to cut the tissue between said rows.

28. A method joining together two opposing walls of a hollow organ, comprising the steps of:
positioning a device inside the hollow organ,
creating at least one opening in each of the two opposing walls of the hollow organ,
passing a jaw of a tissue securement element through each opening, said jaws coupled together at a pivot,
moving the jaws of the tissue securement element together about the pivot to capture the walls of the hollow organ in between,
activating a securement element positioned about the jaws to secure the opposing walls together.

29. The method of claim 28 wherein said hollow organ is the stomach.

30. The method of claim 28 wherein the secured opposing walls form a first partition in the stomach.

31. The method of claim 30 wherein at least a portion of the first partition is positioned near the cardia region of the stomach.

32. The method of claim 30 wherein at least a portion of the first partition is positioned near the lesser curvature region of the stomach.

33. The method of creating at least one opening as in claim 28 further comprising penetrating the organ wall with a puncturing element, wherein said element comprises one or a combination of a needle, knife, radio frequency electrode or dilatation balloon.

34. The method of claim 28 wherein the securement element secures the walls together through the use of one or a combination of staples, t-tags, sutures, clamps, helical anchors or braided anchors.

35. The method of claim 28 including modifying the tissue properties of at least one of the opposing walls prior to activating the securement element.

36. The method of claim 35 wherein the modification includes cutting, piercing, thermal injury, chemical injury, excision or crushing.

37. The method of claim 28 further comprising repositioning said jaws to a different location and moving the jaws of the tissue securement element together about the pivot to capture the walls of the hollow organ in between,
activating the securement element positioned about the jaws to secure the opposing walls together,
wherein the secured opposing walls form a second partition in the stomach.

38. The method of claim 37 wherein said first partition is created near the cardia, thereby forming a pouch, and wherein said second partition is created near the lesser curvature, thereby forming a tubular outlet to the pouch.
